(19) United States
(12) Patent Application Publication
(10) Pub. No.: US 2002/0110585 A1
(43) Pub. Date: Aug. 15, 2002

(54) PATCH THERAPEUTIC AGENT DELIVERY DEVICE HAVING TEXTURIZED BACKING

(76) Inventors: Kristin J. Godbey, Vadnais Heights, MN (US); Daniela Gustin, Heddesheim (DE)

Correspondence Address: OFFICE OF INTELLECTUAL PROPERTY COUNSEL, 3M INNOVATIVE PROPERTIES COMPANY, P O BOX 33427, ST PAUL, MN, 551333427

(21) Appl. No.: 09/451,985

(22) Filed: Nov. 30, 1999

(51) Int. Cl. A61K 9/70; A61F 13/00

(52) U.S. Cl. 424/449

(57) ABSTRACT

A therapeutic agent delivery device has a texturized protective backing, preferably with three-dimensional surface features. By providing the drug delivery device with such a texturized backing, several advantageous properties are imparted to the device, e.g., enhanced ability to camouflage wrinkles and swelling, greater permeability of the backing to water vapor and oxygen, improved flexibility and conformability, and thus, enhanced comfort for the host wearing the device.

### FIELD OF THE INVENTION

This invention relates to a therapeutic agent delivery device of the type that is attached to a host at an administration site in order to therapeutically deliver a drug or other pharmacological agent, cosmeceutical, nutriceutical, or other therapeutically active agent topically, transdermally, transmucosally, or via other transtissue delivery mechanisms. More specifically, this invention relates to such devices that comprise protective backings in which the backing is texturized with three-dimensional surface features. By providing the delivery device with such a texturized backing, several advantageous properties are imparted to the device, e.g., enhanced ability to camouflage wrinkles and swelling, greater permeability of the backing to water vapor and oxygen, improved flexibility and conformability, and thus, enhanced comfort for the host wearing the device.

### BACKGROUND OF THE INVENTION

Delivery of therapeutically active agents via devices (“patch devices”) that are attached to and worn by a host, whether human or animal or plant, is an increasingly important, non-invasive method of agent administration. In practice, a device containing the agent or agents to be administered is placed onto a tissue of a host. The agent, which is releasably stored in a repository of the device, is then caused via diffusional mechanisms or the like to be delivered to the skin of the host in furtherance of the desired therapeutic treatment. Such delivery can be used for topical, transdermal, transmucosal, or other transtissue delivery of the agent and to therapeutically treat local or systemic medical conditions. Patch devices can be used for pharmacological treatments, cosmeceutical treatments, nutriceutical treatments, and/or the like.

A typical patch structure includes a suitable repository that releasably stores an appropriate dosage of one or more therapeutically active agents. The repository layer may be a solid, semi-solid, gel, or liquid. For example, one kind of repository is a liquid in which the therapeutically active agent is dissolved, dispersed, or otherwise distributed. Such liquid repositories are typically stored within a pouch structure incorporated into the patch device. In other patch devices, the repository may be a solid matrix comprising a pressure sensitive adhesive in which the therapeutically active agent is distributed. These and other repository structures are well known and are described, for example, in U.S. Pat. Nos. 5,780,045, 5,851,549, 5,372,819, 5,908,637 and 5,702,720.

In a representative construction, the repository is sandwiched between a protective backing and a release liner. Other layers, such as membrane layers, adhesive layers, barrier layers, or the like, can be incorporated into the device as well. At the time of use, the release liner is easily removed, exposing a tacky surface that allows the patch to be attached to the host. Once attached to the host, the therapeutic agent leaves the repository and diffuses and/or otherwise penetrates into the host, or is topically active, in accordance with the desired therapeutic treatment.

In addition to the therapeutically active agent, the repository may further include one or more other components, such as penetration enhancers that help control the rate at which the drug is administered to the host. Most commonly, penetration enhancers are used to increase the rate of drug delivery inasmuch as the skin or mucosa of a host can be an effective barrier against migration of the drug into the host in therapeutically effective amounts. Other optional ingredients that may be incorporated into the repository include solubilizers, fillers, fragrances, flavorings, stabilizers, and the like.

An optional backing may protect the repository and the components contained in the repository, including the therapeutically active agent, from the environment. The backing also prevents loss of ingredients of the device to the environment. Protective backings may be made from a wide variety of materials conventionally used as backing materials in the medical field, including polymeric films and the like. Polymeric films are widely used, because polymeric materials are generally inert with respect to many components that may be incorporated into a patch device and form excellent barriers to protect the patch components from the environment.

The use of polymeric films as backing materials, however, poses some challenges. First, some ingredients that may be incorporated into a patch device may have a tendency to migrate into the protective backing. When this happens, the protective backing can swell and wrinkle. Examples of ingredients that can cause this problem are some therapeutically active agents, penetration enhancers, solubilizers, and the like. In particular, fatty acids and fatty acid esters are known to cause swelling and wrinkling when used in conjunction with certain polymeric materials. Wrinkling and swelling are not only visually unpleasant, but can also affect the rate of drug delivery if the wrinkling is excessive. It would be desirable to provide a backing material that could reduce or even avoid this problem.

In order to enhance comfort for the host and to promote the health of the tissue underlying a patch device, it may be desirable that the protective backing has reasonably high transmission rates for moisture vapor and/or oxygen. Oxygen transmission is particularly desirable, because this allows the tissue under the patch device to breathe and stay healthy when the patch is worn for long periods. Moisture vapor transmission may be desirable in that moisture has a tendency to build up between the patch and the host, leading to discomfort for the host. Such moisture can also cause the adhesive surface holding the patch to the host to lose its tackiness such that the patch could fall off the host. In such circumstances, it would be desirable that the protective backing have a reasonably high water vapor transmission rate in order to allow moisture to escape the site covered by the transdermal device. Unfortunately, many polymeric films inherently have very low transmission rates for oxygen and moisture vapor. It would be desirable to provide a backing that offers increased transmission rates of oxygen and moisture vapor.

Another issue related to the protective backing of many conventional drug delivery devices concerns flexibility and conformability to the host. For comfort and performance reasons, it is desirable that a backing be able to dynamically conform to the surface of the host at the attachment site, particularly as the host moves. However, many polymeric films are not sufficiently conformable to the complex topographic, moving surfaces of a host. Conformable backings would be highly desired.

### SUMMARY OF THE INVENTION

It has now been discovered that incorporating texturized backings into patch devices imparts several advantageous properties to these drug delivery devices, including the ability to camouflage wrinkling and swelling when the backing comes into contact with some formulation ingredients, increased flexibility/conformability for enhanced comfort, and enhanced permeability to moisture vapor and oxygen. Importantly, one or both surfaces of the backing can be texturized without affecting the bulk physical and mechanical properties of the film used to make the backing. The use of texturizing also expands the variety of different polymeric films that can be used for backings, since many polymeric films can obtain the benefits that texturizing offers. Thus, films that might have acceptable properties in one aspect, but would otherwise be too stiff or too prone to wrinkling and swelling or too impermeable to vapor/oxygen transmission, can be texturized and rendered suitable for use in patch devices.

Thus, in one aspect, the present invention provides a delivery device for use in therapeutically administering a therapeutically active agent, or prodrug form thereof, to a host. In particular, the delivery device comprises a repository comprising a releasably stored dosage of the therapeutically active agent and a texturized backing protecting the repository. The backing comprises inner and outer opposed major surfaces at least one of which is texturized so as to comprise a plurality of three-dimensional topographic features. In one class of preferred embodiments, the inner surface of the backing, the outer surface of the backing, or both, can be texturized with a quantity of three-dimensional surface features effective to improve the ability of the backing to camouflage wrinkles and/or swelling in the backing as compared to an otherwise identical backing lacking such features. In another class of preferred embodiments, either or both surfaces of the backing can be texturized with a quantity of three-dimensional surface features effective to increase the moisture vapor transmission of the backing as compared to an otherwise identical backing lacking such features. In yet another class of preferred embodiments, either or both surfaces of the backing can be texturized with a quantity of three-dimensional surface features effective to impart at least a majority of the surface with a visually discernible texture.

In another aspect, the present invention provides a method of using such a delivery device in order to therapeutically administer one or more therapeutically active agents.

In another aspect, the present invention relates to a method of making such a delivery device. The method involves incorporating a texturized backing into the device.

### BRIEF DESCRIPTION OF THE DRAWINGS

The above mentioned and other advantages of the present invention, and the manner of attaining them, will become more apparent, and the invention itself will be better understood, by reference to the following description of the embodiments of the invention taken in conjunction with the accompanying drawings, wherein:

FIG. 1 is a cross-sectional view of one embodiment of the delivery device in accordance with the present invention, wherein the inner surface of the backing is texturized with an array of microstructured prisms.

FIG. 2A is a cross-sectional view of the texturized backing shown in FIG. 1.

FIG. 2B is a top view of the texturized, inner surface of the backing shown in FIG. 1.

FIG. 3 is a cross-sectional view of a second embodiment of the delivery device in accordance with the present invention, wherein the outer surface of the backing is texturized with an array of microstructured prisms.

FIG. 4 is a cross-sectional view of a third embodiment of the delivery device in accordance with the present invention, wherein both the inner surface and the outer surface of the backing are texturized with respective arrays of microstructured prisms.

FIG. 5A is a perspective view of an alternative embodiment of a backing in accordance with the present invention, wherein a surface of the backing is texturized with a continuous, uniform random texture.

FIG. 5B is a cross-sectional view of the embodiment of the shown in FIG. 5A, wherein a surface of the backing is texturized with a continuous, uniform random texture.

FIG. 6 is a perspective view of a fifth embodiment of the drug delivery device in accordance with the present invention, wherein a surface of the backing is texturized so as to comprise a plurality of rectangular or square boss-like protuberances.

FIG. 7 is a schematic illustration of an apparatus used to evaluate sample swelling.

### DETAILED DESCRIPTION

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

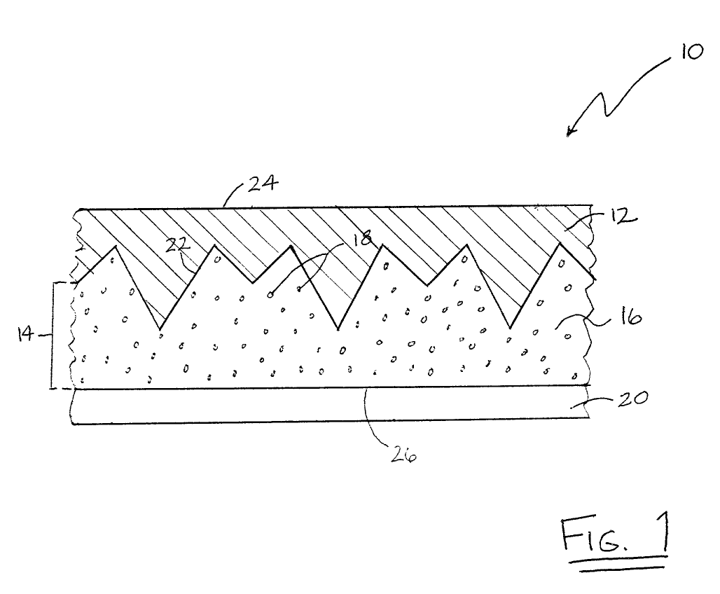

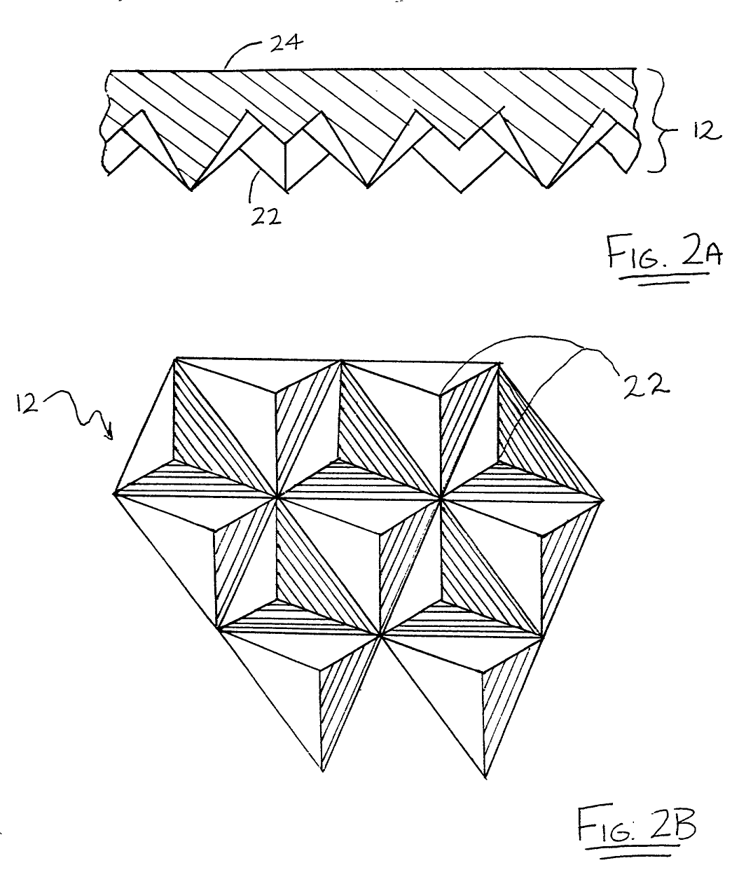

The principles of the present invention can be incorporated into a wide variety of different drug delivery devices designed to be attached to a host for topical, transdermal, transmucosal, or other transtissue delivery of one or more therapeutically active agents. As one representative embodiment of such a structure, the present invention will now be described in connection with the drug delivery device 10 illustrated in FIGS. 1, 2A and 2B. Device 10 generally includes backing 12, repository layer 14, release liner 20 and therapeutically active agent 18 contained within repository layer 14. Backing 12 includes outer major surface 24 and inner major surface 22.

Backing 12 may be formed from any conventional backing material. Preferably, backing 12 is formed from any flexible material that protects repository layer 14 from the environment, resists bulk fluid flow, provides a barrier against loss of therapeutically active agent 18, and is substantially chemically inert with respect to the ingredients incorporated into the other components of patch device 10. Backing 12, for instance, may be formed from one or more conventional materials typically used as backings for tapes, bandages, wound dressings, other transtissue delivery devices, and the like. In the case of a composition that contains a therapeutically active agent intended to be delivered across a membrane such as skin or a mucosal membrane and intended to have systemic action, the backing is preferably substantially resistant to the migration of the therapeutically active agent therethrough. In the case of a composition that contains a therapeutically active agent intended to be delivered, e.g. to the oral cavity or the vaginal cavity and/or intended to have local action, the backing can be permeable to the agent to be delivered and can be permeable to saliva as well. With respect to embodiments of the invention in which therapeutically active agent 18 is stored in a fluid reservoir, it may be desirable that backing 12 is heat sealable to itself and to a variety of other polymeric materials at relatively low temperatures.

Representative examples of suitable backing materials include polyethylene, polypropylene, ethylene-vinyl acetate copolymers, polyurethane, ethylene propylenediene copolymer, polyisobutylene, other polyolefins, polyamide, polyester, copolymers of two or more monomers, combinations of these, and the like. Preferred backing materials include an acrylate pressure-sensitive adhesive coated polyurethane film such as TEGADERM brand transparent dressing (commercially available from the Minnesota Mining and Manufacturing Company (3M), St. Paul, Minn.). Specific embodiments of suitable backing materials are further described in U.S. Pat. No. 5,372,819, the entire disclosure of which is incorporated by reference herein for all purposes.

Many such polymeric materials that may be used to form backing 12 may have a tendency to wrinkle and/or swell when ingredients incorporated into drug delivery device 10 come into contact with, migrate or are otherwise transported into backing 12. For instance, in those embodiments of the invention where repository layer 14 comprises penetration enhancers such as fatty acid and/or fatty acid ester constituents, these penetration enhancers may migrate into and cause wrinkling and swelling of backing 12. Even if migration of other components into backing 12 is not a concern, films comprised of some of the aforementioned polymeric materials may show poor conformability to the surface of the host to which drug delivery device 10 might be attached. This can be uncomfortable for the host and/or can result in drug delivery device 10 falling off of the host. Some films also form barriers that prevent water vapor and/or oxygen from effectively leaving the host site to which device 10 is attached. This, too, can be uncomfortable for the host or aesthetically unpleasant in appearance.

Accordingly, the present invention advantageously provides texturized features on one or both of inner surface 22 and/or outer surface 24 of backing 12 in order to minimize or camouflage wrinkling and swelling, improve the conformability of backing 12 to the contour of the host to which it is ultimately applied, and/or improve water vapor and/or oxygen transmission through backing 12. As shown, only inner surface 22 is textured in the illustrated environment. However, as desired, the texture may be beneficially formed on, or into, one or both of surfaces 22 and 24 using an appropriate texturizing technique to form a plurality of three-dimensional topographic features. In one approach, this may be achieved by incorporating particles, fibers, or other three dimensional structures, which may be organic or inorganic, into the texturized surface such that the particles form protuberances that project outward from the surface. In those applications where it would be desirable to avoid incorporating extraneous ingredients into backing 12, one or both surfaces 22 and 24 of backing 12 are preferably texturized with three-dimensional features that are integrally formed with backing 12 itself. One or both of surfaces 22 and 24 can be so texturized using a wide variety of techniques well known in the art. Examples of such techniques include cold or hot embossing with patterned surfaces (plates, rollers, or the like), extrusion casting onto a patterned surface, flame embossing, corona discharge treatment, light e-beam treatment, ultraviolet irradiation, etching, ablating, combinations of these, or the like.

The texture imparted onto one or both of surfaces 22 and 24 is not particularly restricted, but rather may comprise protuberances and/or depressions having a wide variety of shapes, dimensions, spacing, and arrangement. For example, the texture may comprise features in the shape of rectilinear prisms, linear or curved grooves or ridges, acicular bumps or depressions, tapering or nontapering posts or holes, combinations of these, and the like. For purposes of illustration, FIGS. 1-4 show inner surface 22 and/or outer surface 24 bearing a texture comprising a plurality of features in the form of a microstructured array of trihedral prisms.

The features on the texturized surface of backing 12 can have a wide range of suitable dimensions. In some embodiments, the features may be so small that individual features are not visually discernible to the naked eye, yet the aggregate of the features provides the texturized surface with a visually discernible matte finish. Alternatively, the features may be large enough so that individual features may be visually seen by the naked eye. However, if the features are too small or too large, the texturized surface may not adequately camouflage wrinkles and swelling, improve flexibility, improve vapor or O₂ transmission, and/or the like, as may be desired. Preferred features have a height or depth, as the case may be, in the range of from about 50 micrometers to 1000 micrometers, more preferably from about 100 to about 250 micrometers.

The arrangement of features on the texturized surface may be random or regular and may cover substantially all or only a portion of surface 22 and/or 24 as desired. Preferably, at least 50%, more preferably at least 75%, and most preferably at least substantially all of inner surface 22 and/or outer surface 24 is texturized. The features may be adjacent to one another, as is the case with the prisms 22 in FIG. 2B or alternatively, the features may be spaced apart from each other. However, if spaced apart from one another, the spacing should not be so great that the camouflage, flexibility, and/or permeability characteristics are unduly compromised. As suggested guidelines, the spacing between features preferably is no more than about five times, preferably no more than 2 to 3 times, the breadth of the individual features.

Specific examples of the shapes, dimensions, spacing, and arrangement of topographic features that may be advantageously imparted onto one or both of surfaces 22 or 24 on backing 12 of the drug delivery device of the present invention are further described in U.S. Pat. Nos. 5,508,084; 5,454,844; 5,152,917; 4,799,054; and 4,055,029.

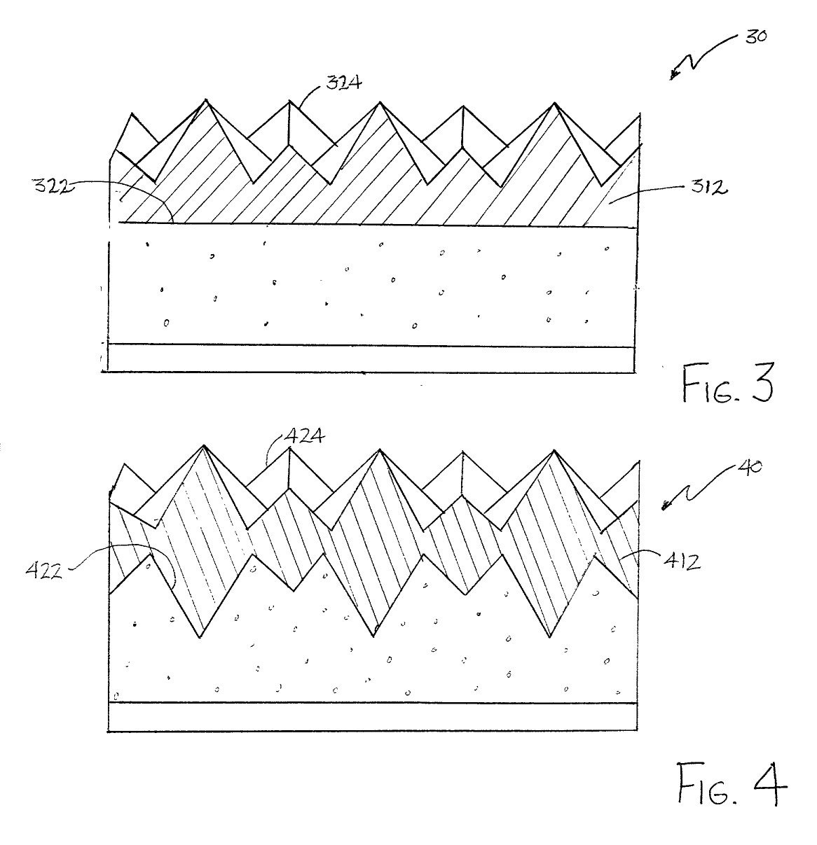

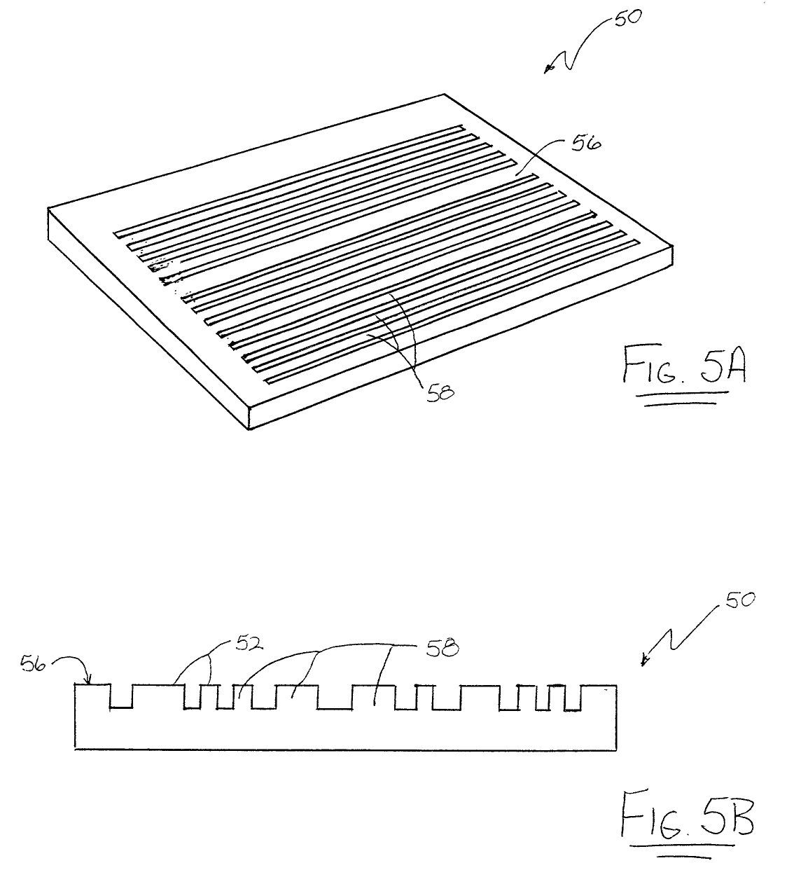

As mentioned hereinabove, either one or both of inner and outer surface of the backing layer may be texturized, as is further illustrated by FIGS. 3 and 4. Specifically, FIG. 3 illustrates drug delivery device 30 having a texturized backing 312 wherein the outer surface 324 is texturized, while FIG. 4 illustrates drug delivery device 40 having a texturized backing 412 wherein both the outer surface 424 and the inner surface 422 are texturized.

Referring again to FIGS. 1, 2A and 2B, backing 12 serves, at least in part, to protect repository layer 14 from the environment. Repository layer 14 comprises a dosage of therapeutically active agent 18 stored in matrix 16. As illustrated, matrix 16 is a solid drug repository comprising a pressure sensitive adhesive so that surface 26 is tacky and can be used to attach patch device 10 to a host. Of course, the repository layer used in the present invention need not be a solid matrix comprising a pressure sensitive adhesive, but may also comprise another solid formulation, a gel, a semi-solid matrix, a fluid reservoir, and the like. Examples of other kinds of repository compositions and structures are described in U.S. Pat. Nos. 4,814,173; 4,834,979; 4,820,525; 5,310,559.

One preferred class of materials that is generally capable of forming matrix 16 is pressure sensitive adhesives. A wide variety of pressure sensitive adhesives may be used to form matrix 16 of repository layer 14. Desirably, the particular pressure sensitive adhesive material to be used is a solid or semi-solid and is at least substantially chemically inert with respect to the other components of drug delivery device 10, particularly the therapeutically active agent 18, or prodrug form thereof, if any. For therapeutic applications, the pressure sensitive adhesive material also should adhere well to the desired treatment site of the host animal. Preferably, the pressure sensitive adhesive material is water resistant so that drug delivery device 10 remains adhered to the host for the desired treatment period even when exposed to moisture, but should be releasable so that drug delivery device 10 can be removed when desired. The pressure sensitive adhesive material should also be compatible with the host so that undue irritation at the treatment site is avoided. Furthermore, the pressure sensitive adhesive material preferably is sufficiently flexible to allow drug delivery device 10 to conform to and follow the contours of the treatment site without cracking and without causing undue restriction of host movement.

Suitable classes of pressure sensitive adhesive materials meeting these criteria include silicone, polyisobutylene polymers and (meth)acrylate polymers, and in particular, the acrylate embodiments thereof. Representative embodiments of such (meth)acrylate pressure sensitive adhesives are described in U.S. Pat. Nos. 4,751,087; 4,737,577; 4,737,559; 4,693,776; and Re 24,906; and in PCT WO 96/08229, the entire disclosures of each being incorporated herein by reference for all purposes.

A (meth)acrylate pressure sensitive adhesive suitable in the practice of the present invention preferably has a weight average molecular weight that is high enough so that the polymer has good handling, performance, and mechanical properties. However, if the weight average molecular weight of the (meth)acrylate pressure sensitive adhesive is too high, fluid compositions incorporating such adhesive may have a viscosity that is too high. Viscosity properties are of concern during manufacture of drug delivery device 10 when repository layer 14 is formed from a solution or dispersion of ingredients including the pressure sensitive adhesive materials. Accordingly, a preferred (meth)acrylate pressure sensitive adhesive generally has a weight average molecular weight in a range such that the adhesive has an inherent viscosity in the range from about 0.2 dL/g to about 2 dL/g, more preferably from about 0.4 dL/g to about 1.4 dL/g. Inherent viscosity may be determined by conventional means using a Canon-Fenske #50 viscometer in a water bath controlled at 27° C. to measure the flow of 10 mL of polymer solution.

A particularly preferred (meth)acrylate pressure sensitive adhesive is a copolymer derived from monomers comprising, based upon the total weight of the copolymer, about 40 to about 100, preferably about 50 to about 75, weight percent of an alkyl (meth)acrylate (A monomer) and 0 to about 60, preferably about 25 to about 50, weight percent of a free radically copolymerizable monomer (B monomer). Optionally, other monomers may also be incorporated into the copolymers. Such other monomers, for example, may further include up to about 30 weight percent, preferably up to about 15 weight percent, of a copolymerizable macromonomer as described in PCT publication WO 96/08229.

The A monomer preferably is selected from one or more alkyl (meth)acrylates containing 1 to about 10 carbon atoms in the alkyl group. Representative examples of the alkyl (meth)acrylate monomer include methyl (meth)acrylate, n-butyl (meth)acrylate, n-pentyl (meth)acrylate, n-hexyl (meth)acrylate, isoheptyl (meth)acrylate, cyclohexyl (meth)acrylate, n-nonyl (meth)acrylate, n-decyl (meth)acrylate, isohexyl (meth)acrylate, 2-ethyloctyl (meth)acrylate, isooctyl (meth)acrylate, isobornyl (meth)acrylate, and 2-ethylhexyl (meth)acrylate. Combinations of these can be used if desired. Preferably, the alkyl (meth)acrylate is selected from isooctyl (meth)acrylate, butyl methacrylate, 2-ethylhexyl (meth)acrylate, cyclohexyl methacrylate, isobornyl methacrylate, and methyl methacrylate.

The copolymerizable B monomer is generally one or more (meth)acrylate monomers having at least one functional group selected from the grouping consisting of carboxylic acid, carboxylic acid ester, hydroxyl, anydride, epoxy, thiol, isocyanate, sulfonamide, urea, carbamate, carboxamide, amine, ammonium, oxy, oxo, nitro, nitrogen, sulfur, phosphate, phosponate, cyano, combinations of these, and the like. Representative examples of specific materials that can be used singly or in combination as the B monomer include (meth)acrylic acid, maleic acid, vinyl acetate, a hydroxyalkyl (meth)acrylate containing about 2 to about 4 carbon atoms in the hydroxyalkyl group, (meth)acrylamide, an alkyl substituted (meth)acrylamide having 1 to about 8 carbon atoms in the alkyl group, diacetone (meth)acrylamide, a dialkyl (meth)acrylamide independently having 1 or 2 carbon atoms in each alkyl group, N-vinyl-N-methyl acetamide, N-vinyl valerolactam, N-vinyl caprolactam, N-vinyl-2-pyrrolidone, glycidyl (meth)acrylate, alkoxy (meth)acrylate containing 1 to 4 carbon atoms in the alkoxy group, 2-ethoxyethyl (meth)acrylate, 2,2-ethoxyethoxyethyl (meth)acrylate, furfuryl (meth)acrylate, tetrahydrofurfuryl (meth)acrylate, propylene glycol mono(meth)acrylate, polyethylene glycol (meth)acrylate, polyethylene glycol methyl ether (meth)acrylate, polyethylene oxide methyl ether (meth)acrylate, di(lower)alkylaminopropyl (meth)acrylamide (wherein lower means the alkyl moiety has 1 to 4 carbon atoms), (meth)acrylonitrile, combinations of these, and the like. Preferably, the copolymerizable B monomer is selected from hydroxyethyl acrylate, hydroxyethyl methacrylate, acrylamide, glyceryl acrylate, N,N-dimethyl acrylamide, 2-ethoxyethyl acrylate, 2,2-ethoxyethoxyethyl acrylate, tetrahydrofurfuryl acrylate, vinyl acetate, and acrylic acid. Any of the aforementioned alkyl groups may be linear, branched or cyclic.

One particularly preferred (meth)acrylate pressure sensitive adhesive is a copolymer formed by copolymerizing about 60 to about 80, preferably about 75 weight percent of isooctyl (meth)acrylate (preferably the acrylate form); about 1 to about 10, preferably about 5 weight percent of (meth)acrylamide (preferably the acrylate form); and about 5 to about 30, preferably about 20 weight percent of vinyl acetate. This (meth)acrylate pressure sensitive adhesive demonstrates excellent adhesion to the skin of a human or other animal host, is flexible and waterproof, is soluble in therapeutically compatible solvents such as isopropyl alcohol, and is very compatible with many kinds of therapeutically active agents. Other preferred (meth)acrylate pressure sensitive adhesive polymers are formed from monomers according to formulations summarized in the following table:

| | Parts by weight: | | | | | | |
|---|---|---|---|---|---|---|---|
| PSA | IOA | ACM | VOAc | DMACM | AA | HEA | NVP |
| 1 | 93 | 7 | — | — | — | — | — |
| 2 | 70 | — | — | 30 | — | — | — |
| 3 | 63 | — | 37 | — | — | — | — |
| 4 | 80 | — | — | — | 20 | — | — |
| 5 | 60 | — | — | — | — | 40 | — |
| 6 | 91 | — | — | — | — | — | 9 |
| 7 | 89 | — | — | — | — | 2 | 9 |

wherein IOA is isooctyl acrylate, ACM is acrylamide, VOAc is vinyl acetate, DMACM is N,N-dimethylacrylamide, AA is acrylic acid, HEA is 2-hydroxyethyl acrylate, and NVP is N-vinylpyrrolidone.

The particularly preferred (meth)acrylate pressure sensitive adhesive may be prepared by free-radical polymerization methods known in the art, including but not limited to bulk, solution, emulsion and suspension polymerization methods. For example, according to the solution polymerization method, copolymers suitable for use in the present invention are prepared by dissolving the desired monomers in an appropriate solvent, adding a chain-transfer agent, a free-radical polymerization initiator, and other additives known in the art, sealing the solution in an inert atmosphere such as nitrogen or argon, and then agitating the mixture at a temperature sufficient to activate the initiator.

Solvents useful in such polymerizations can vary according to solubility of the monomers and additives. Typical solvents include ketones such as acetone, methyl ethyl ketone, 3-pentanone, methyl isobutyl ketone, disobutyl ketone, and cyclohexanone; alcohols such as methanol, ethanol, propanol, n-butanol, isopropanol, isobutanol, cyclohexanol and methyl cyclohexanol; esters such as ethyl acetate, butyl acetate, isobutyl acetate, isopropyl acetate, and the like; aromatic hydrocarbons such as benzene, toluene, xylenes, cresol, and the like; ethers such as diisopropyl ether, diisobutyl ether, tetrahydrofuran, tetrahydropyran, and dioxane; and aprotic solvents such as dimethylformamide, dimethylsulfoxide and the like, and mixtures thereof.

Chain transfer agents suitable for solution polymerization include but are not limited to alcohols, mercaptans, certain halogenated small molecules, and mixtures thereof. Preferably, the chain transfer agent is chosen from the group consisting of carbon tetrabromide, isooctylthioglycolate, mercaptosuccinic acid, mercaptopropane diol, dodecyl mercaptan, ethanol and carbon tetrachloride. Most preferably, the chain transfer agent is mercaptopropane diol.

Free-radical polymerization initiators suitable for solution polymerization include those that are soluble in the reaction solvent and that are thermally activated, including but not limited to azo compounds, peroxides, and mixtures thereof. Useful peroxide initiators include those chosen from the group consisting of benzoyl peroxide, lauroyl peroxide, di-t-butyl peroxide and the like, and mixtures thereof. Useful azo compound initiators include those chosen from the group consisting of 2,2′-azobis (2-methylbutyronitrile); 2,2′azobis (isobutyronitrile); and 2,2′-azobis (2,4-dimethylpentanenitrile); each of which is commercially available as VAZO 67, VAZO 64, and VAZO 52, respectively, from E. I. DuPont de Nemours & Co., Wilmington, Del.

The (meth)acrylate pressure sensitive adhesive polymers suitable in the practice of the present invention may also be prepared by emulsion polymerization methods. According to the emulsion polymerization method, polymers are prepared by forming an emulsion comprising the desired monomers, a chain-transfer agent and a water-soluble redox-type initiator system in an inert atmosphere such as nitrogen or argon, and then heating the emulsion carefully until a reaction exotherm occurs., The reaction mixture is stirred and cooled and the resulting polymer is collected. Optionally, an ionic or nonionic surfactant may be added to the reaction mixture. Oxidation-reduction (“Redox”) free-radical initiators may also optionally be added. Redox initiators include, but are not limited to, those chosen from the group consisting of tertiary amines with organic peroxides (exemplified by the N,N-diethylaniline-benzoyl peroxide pair); organic halides with transition metal complexes (exemplified by the carbon tetrachloride-molybdenum hexacarbonyl pair); inorganic oxidation-reduction systems (exemplified by the potassium persulfate-sodium metabisulfite pair); and organic-inorganic systems (exemplified by the 2-mercaptoethanol-Fe⁺³ pair). Inorganic redox initiators are preferred because of their ease of handling and useful reaction temperature range.

In addition to matrix 16, repository layer 14 comprises an amount of one or more therapeutically active agent(s) 18. The particular therapeutically active agent 18 employed is not critical, but rather will depend upon the end use of drug delivery device 10. Representative examples of therapeutically active agents that may be suitable for use in drug delivery device 10 of the present invention include the following (grouped by therapeutic class) as well as prodrugs thereof:

Antidiarrhoeals such as diphenoxylate, loperamide and hyoscyamine;

Antihypertensives such as hydralazine, minoxidil, captopril, enalapril, clonidine, prazosin, debrisoquine, diazoxide, guanethidine, methyldopa, reserpine, trimethaphan;

Calcium channel blockers such as diltiazem, felodipine, amlodipine, nitrendipine, nifedipine and verapamil;

Antiarrhyrthmics such as amiodarone, flecainide, diisopyramide, procainamide, mexiletene and quinidine;

Antiangina agents such as glyceryl trinitrate, erythrityl tetranitrate, pentaerythritol tetranitrate, mannitol hexanitrate, perhexilene, isosorbide dinitrate and nicorandil;

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate;

Cardiotonic glycosides such as digoxin and other cardiac glycosides and theophylline derivatives;

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine;

Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol;

Antimigraine preparations such as ergotamine, dihydroergotamine, methysergide, pizotifen and sumatriptan;

Anticoagulants and thrombolytic agents such as warfarin, dicoumarol, low molecular weight heparins such as enoxaparin, streptokinase and its active derivatives;

Hemostatic agents such as aprotinin, tranexamic acid and protamine;

Analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenoperidine, codeine dihydrocodeine; acetylsalicylic acid (aspirin), paracetamol, and phenazone;

Hypnotics and sedatives such as the barbiturates amylobarbitone, butobarbitone and pentobarbitone and other hypnotics and sedatives such as chloral hydrate, chlormethiazole, hydroxyzine and meprobamate;

Antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam;

Neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium;

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline;

CNS stimulants such as caffeine, 3-(2-aminobutyl) indole, and methylphenidate (Ritalin);

Anti-alzheimer's agents such as tacrine;

Anti-Parkinson's agents such as amantadine, benserazide, carbidopa, levodopa, benztropine, biperiden, benzhexol, procyclidine and dopamine-2 agonists such as S(−)-2-(N-propyl-N-2-thienylethylamino)-5-hydroxytetralin (N-0923);

Anticonvulsants such as phenytoin, valproic acid, primidone, phenobarbitone, methylphenobarbitone and carbamazepine, ethosuximide, methsuximide, phensuximide, sulthiame and clonazepam;

Antiemetics and antinauseants such as the phenothiazines prochloperazine, thiethylperazine and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride;

Non-steroidal anti-inflammatory agents including their racemic mixtures or individual enantiomers where applicable, preferably which can be formulated in combination with dermal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate;

Antirheumatoid agents such as penicillamine, aurothioglucose, sodium aurothiomalate, methotrexate and auranofin;

Muscle relaxants such as baclofen, diazepam, cyclobenzaprine hydrochloride, dantrolene, methocarbamol, orphenadrine and quinine;

Agents used in gout and hyperuricaemia such as allopurinol, colchicine, probenecid and sulphinpyrazone;

Oestrogens such as oestradiol, oestriol, oestrone, ethinyloestradiol, mestranol, stilboestrol, dienoestrol, epioestriol, estropipate and zeranol;

Progesterone and other progestagens such as allyloestrenol, dydrogesterone, lynoestrenol, norgestrel, norethyndrel, norethisterone, norethisterone acetate, gestodene, levonorgestrel, medroxyprogesterone and megestrol;

Antiandrogens such as cyproterone acetate and danazol;

Antioestrogens such as tamoxifen and epitiostanol and the aromatase inhibitors, exemestane and 4-hydroxy-androstenedione and its derivatives;

Androgens and anabolic agents such as testosterone, methyltestosterone, clostebol acetate, drostanolone, furazabol, nandrolone, oxandrolone, stanozolol, trenbolone acetate, dihydro-testosterone, 17-α-methyl-19-nortestosterone and fluoxymesterone;

5-Reductase inhibitors such as finasteride, turosteride, LY-191704 and MK-306;

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide;

Further examples of steroidal antiinflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, flucetonide, fludrocortisone acetate, betamethasone benzoate, chloroprednisone acetate, clocortolone acetate, descinolone acetonide, desoximetasone, dichlorisone acetate, difluprednate, flumethasone pivalate, flunisolide acetate, fluperolone acetate, fluprednisolone valerate, paramethasone acetate, prednisolamate, prednival, triamcinolone hexacetonide, cortivazol, formocortal and nivazol;

Pituitary hormones and their active derivatives or analogs such as corticotrophin, thyrotropin, follicle stimulating hormone (FSH), luteinising hormone (LH) and gonadotrophin releasing hormone (GnRH);

Hypoglycemic agents such as insulin, chlorpropamide, glibenclamide, gliclazide, glipizide, tolazamide, tolbutamide and metformin;

Thyroid hormones such as calcitonin, thyroxine and liothyronine and antithyroid agents such as carbimazole and propylthiouracil;

Other miscellaneous hormone agents such as octreotide;

Pituitary inhibitors such as bromocriptine;

Ovulation inducers such as clomiphene;

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potasium sparing diuretics, spironolactone, amiloride and triamterene;

Antidiuretics such as desmopressin, lypressin and vasopressin including their active derivatives or analogs;

Obstetric drugs including agents acting on the uterus such as ergometrine, oxytocin and gemeprost;

Prostaglandins such as alprostadil (PGE1), prostacyclin (PGI2), dinoprost (prostaglandin F2-alpha) and misoprostol;

Antimicrobials including the cephalosporins such as cephalexin, cefoxytin and cephalothin;

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin;

Tetracyclines such as minocycline, chlortetracycline, tetracycline, demeclocycline, doxycycline, methacycline and oxytetracycline and other tetracycline-type antibiotics;

Aminoglycosides such as amikacin, gentamicin, kanamycin, neomycin, netilmicin and tobramycin;

Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione;

Quinolones such as nalidixic acid, cinoxacin, ciprofloxacin, enoxacin and norfloxacin;

Sulphonamides such as phthalysulphthiazole, sulfadoxine, sulphadiazine, sulphamethizole and sulphamethoxazole;

Sulphones such as dapsone;

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; and benzoylperoxide;

Antituberculosis drugs such as ethambutol, isoniazid, pyrazinamide, rifampicin and clofazimine;

Antimalarials such as primaquine, pyrimethamine, chloroquine, hydroxychloroquine, quinine, mefloquine and halofantrine;

Antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine;

Anthelmintics such as mebendazole, thiabendazole, niclosamide, praziquantel, pyrantel embonate and diethylcarbamazine;

Cytotoxic agents such as plicamycin, cyclophosphamide, dacarbazine, fluorouracil and its prodrugs (described, for example, in International Journal ofPharmaceutics 111, 223-233 (1994)), methotrexate, procarbazine, 6-mercaptopurine and mucophenolic acid;

Anorectic and weight reducing agents including dexfenfluramine, fenfluramine, diethylpropion, mazindol and phentermine;

Agents used in hypercalcaemia such as calcitriol, dihydrotachysterol and their active derivatives or analogs;

Antitussives such as ethylmorphine, dextromethorphan and pholcodine;

Expectorants such as carbolcysteine, bromhexine, emetine, quanifesin, ipecacuanha and saponins;

Decongestants such as phenylephrine, phenylpropanolamine and pseudoephedrine;

Bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives;

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine;

Local anaesthetics such as bupivacaine, amethocaine, lignocaine, lidocaine, cinchocaine, dibucaine, mepivacaine, prilocaine; etidocaine; and procaine;

Stratum corneum lipids, such as ceramides, cholesterol and free fatty acids, for improved skin barrier repair [Man, et al., J. Invest. Dermatol., 106(5), 1096, (1996)];

Neuromuscular blocking agents such as suxamethonium bromide, alcuronium dichloride, pancuronium bromide, atracurium besylate, gallamine, tubocurarine and vecuronium;

Smoking cessation agents such as nicotine, bupropion and ibogaine;

Insecticides and other pesticides which are suitable for local or systemic application;

Dermatological agents, such as vitamins A, C, B₁, B₂, B₆, B₁₂ₐ and E, vitamin E acetate and vitamin E sorbate; salts and esters of such vitamins; and provitamin forms.

Allergens for desensitisation such as house, dust or mite allergens;

Homeopathic agents;

Nutritional agents, such as vitamins, essential amino acids and essential fats;

Keratolytics such as the alpha-hydroxy acids, glycolic acid and salicylic acid;

Anti-acne agents such as isotretinoin, tretinoin and benzoyl peroxide;

Anti-psoriasis agents such as etretinate, cyclosporin and calcipotriol;

Anti-itch agents such as capsaicin and its derivatives such as nonivamide [Tsai, et al., Drug. Dev. Ind. Pharm., 20(4), 719, 1994];

Anticholinergic agents, which are effective for the inhibition of axillary sweating and for the control of prickly heat;

Antiperspirant agents such as methatropine nitrate, propantheline bromide, scopolamine, methscopolamine bromide, and quaternary acyloxymethyl ammnonium salts (described, for example, by Bodor et al., J. Med. Chem. 23, 474 (1980) and also in United Kingdom Specification No. 2010270, published Jun. 27, 1979); and

Other pharmacologically active small to medium-sized peptides and proteins, e.g., vasopressin and human growth hormone and enzymes.

Fragrances, aromatherapy, and cosmeceutical agents include anti-wrinkle agents, anti-baldness agents, moisturizers, sunscreens, antiperspirants, and the like.

The amount of the therapeutically active agent is not particularly restricted but rather, may be limited by practical concerns. For example, if too much therapeutically active agent 18 is present, the adhesive properties of pressure sensitive matrix 16 may be reduced such that the pressure sensitive adhesive matrix 16 is not tacky enough to be attached to a host at the desired administration site. Also, therapeutically active agent 18 may be administered at too high a dosage rate if too much therapeutically active agent 18 is present. On the other hand, if too little of the therapeutically active agent 18 is present, then the rate at which the therapeutically active agent 18 is administered to the host may be too low.

The amount of the particular therapeutically active agent 18 to be included in repository layer 14 can be selected in accordance with conventional practices, and will be dependent upon the particular therapeutically active agent or combination of therapeutically active agents used, the intended therapy, the characteristics of the intended host(s), the desired duration of the treatment, the length of time that the particular device 10 can be worn, and the like. Bearing these general guidelines in mind, repository layer 14 may include 0.01 weight percent to about 40 weight percent of one or more therapeutically active agents 18 based upon the total weight of repository layer 14.

Optionally, in addition to the therapeutically active agent 18 and matrix 16, repository layer 14 may include other optional ingredients. One example of a preferred optional ingredient that is advantageously incorporated into repository layer 14 is one or more penetration enhancers. A penetration enhancer is an agent that improves the transtissue penetration rate of therapeutically active agent 18 through or into a tissue such as skin, a mucosal membrane, or other tissue, whether such transtissue delivery is intended for local or systemic delivery.

If a penetration enhancer is to be included in repository layer 14, it is preferably included in an amount sufficient to cause delivery to occur at the desired rate. The amount of a penetration enhancer required to achieve such an objective can be determined by one skilled in the art in accordance with conventional practices. In determining a suitable amount of penetration enhancer to be used, the skilled worker would give due consideration to factors such as the nature of the other ingredients of the delivery device, the nature of the penetration enhancer, the age and weight of the host, the nature of the host surface to which delivery device 10 will be applied, and the like. As general guidelines, preferred delivery devices of the present invention include about 1 part by weight to about 50 parts by weight, preferably about 5 parts by weight to about 40 parts by weight, more preferably about 10 parts by weight to about 30 parts by weight of the penetration enhancer per 100 parts by weight of repository layer 14.

Representative examples of penetration enhancers include esters of the type described in PCT Publication WO 97/29735, laurocaprolactone and its derivatives such as 1-alkylazacycoheptan-2-ones as described in U.S. Pat. No. 5,196,410; oleic acid and its ester derivates such as methyl oleate, ethyl oleate, propyl oleate, isopropyl oleate, butyl oleate, vinyl oleate, and glyceryl monooleate; sorbitan esters such as sorbitan monolaurate and sorbitol monooleate; other fatty acid esters such as glyceryl monolaurate, isopropyl laurate, isopropyl myristate, isopropyl palmitate, diisopropyl adipate, propylene glycol monolaurate, and propylene glycol monooleate; long chain alkyl esters of 2-pyrrolidone, such as 1-lauryl, 1-hexyl, and 1-(2-ethylhexyl)esters of 2-pyrrolidone; a penetration enhancer of the type described in U.S. Pat. No. 5,082,866 such as dodecyl (N,N-dimethylamino)acetate and dodecyl (N,N-dimethylamino)propionate; a penetration enhancer as described in U.S. Pat. No. 4,861,764 such as 2-n-nonyl-1-3-dioxolane; combinations of these, and the like. A specific example of a combination penetration enhancer is one that includes 10 to 70 parts by weight of isopropyl myristate, about 1 to about 25 parts by weight of glyceryl monolaurate, and about 5 to about 70 parts by weight of ethyl oleate per 100 parts by weight of the penetration enhancer.

Suitable penetration enhancers may also include anionic surfactants (sodium lauryl sulfate), cationic surfactants (cetylpyridinium chloride), nonionic surfactants (polysorbate 80, polyoxyethylene 9-lauryl ether, glyceryl monolaurate), bile salts and related compounds (sodium glycocholate, sodium taurocholate, sodium tauro-24,25-dihydrofusidate), combinations of these, and the like. Such penetration enhancers are also listed in U.S. Pat. Nos. 5,688,520 and 5,908,637.

In addition to or in place of, one or more penetration enhancers, repository layer 14 may also include other ingredients such as flavorings, flavor masking agents, water soluble or water swellable fibrous reinforcers, fragrances, odor-masking agents coloring agents, solubilizers, solvent, fillers, antistatic agents, plasticizers, antioxidants, combinations of these, and the like.

To protect repository layer 14 prior to use, repository layer 14 of delivery device 10 is generally releasably covered on the surface of repository layer 14 opposite to backing 12 with protective release liner 20 in the form of a film or a foil. Suitable release liners for use in the above-described methods of preparation include conventional release liners comprising a known sheet material, such as a polyester film, polyolefin film, polystyrene film, or a polyolefin-coated paper bearing a suitable silicone-type coating, such as Daubert 164-Z (commercially available from Daubert Co., Elmhurst, Ill., or a fluoropolymer-coated release liner, such as SCOTCHPAK™ 1022 brand available from 3M.

In preferred embodiments, backing 12 and/or release liner 14 may optionally incorporate a multilayer optical film. The use of such films in patch drug devices is described in assignee's application filed co-currently herewith in the names of Godbey et al. and having Attorney Docket No. 54911USA9A and titled PATCH THERAPEUTIC AGENT DELIVERY DEVICE INCORPORATING MULTILAYER OPTICAL FILM.

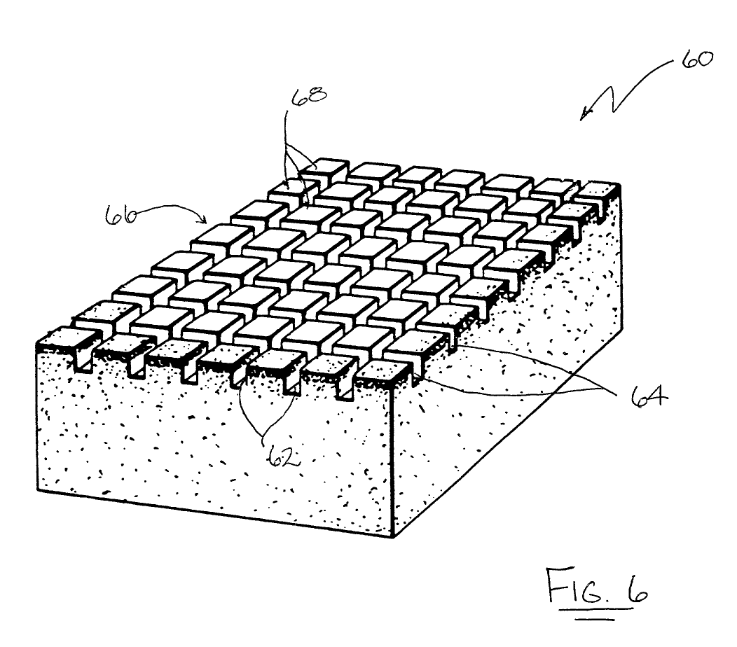

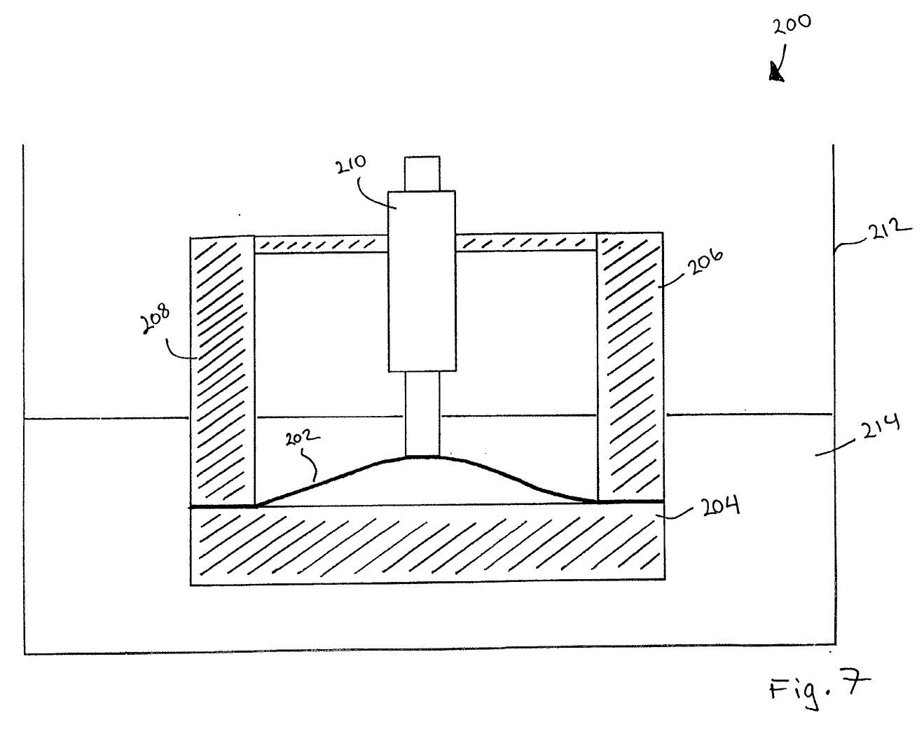

As mentioned hereinabove, backing layer 12 may be texturized in any manner, i.e., with any shapes or features. Exemplary alternative texturized backing layers are shown in FIGS. 5A, 5B and 6. Particularly, FIGS. 5A and 5B show a perspective and cross-sectional view, respectively, of a backing layer 50 suitable for use in the delivery device of the present invention wherein surface 56 of backing layer 50 has been texturized in a manner commonly referred to as a continuous, uniform random texture. As shown in FIGS. 5A and 5B, this texture generally comprises a relatively large number of plateau-like ridges 58 scattered at random on the surface and each having a top 52 at approximately the same height.

Referring now to FIG. 6, there is illustrated yet another backing layer 60 suitable for use in the delivery device of the present invention wherein surface 66 thereof has been texturized to comprise a plurality of parallel grooves 62 and parallel grooves 64. Parallel grooves 62 intersect parallel grooves 64 at an angle of 90°. Consequently, rectangular or square boss-like protuberances 68 are formed in surface 66 of backing layer 60. The height of each of protuberances 68 is illustrated as being generally equal to the depth of grooves 62 and 64. Grooves 62 and 64 have a depth and breadth which is generally smaller than the edge dimensions of bosses 68.

The drug delivery devices in accordance with the present invention can be prepared by general methods well known to those skilled in the art. Embodiments in which the drug repository is a pressure sensitive adhesive matrix generally can be prepared, for instance, using methods set forth in U.S. Pat. Nos. 5,688,523; 4,714,655; 5,059,189; 5,264,224. Those embodiments specifically intended to be used for transmucosal delivery and including a matrix in which the adhesive is a mucoadhesive can be made by methods set forth in WO 90/06505. When the repository is a gel, representative manufacturing methods are set forth in U.S. Pat. Nos., U.S. Pat. No. 4,834,979, EP 556,158. Devices including a fluid reservoir in which the therapeutically active agent is stored may be prepared by the representative methods set forth in U.S. Pat. Nos. U.S. Pat. No. 4,834,979, EP 556,158.

The therapeutic delivery device of the present invention is easily used. Specifically, release liner 20 is removed and drug delivery device 10 applied directly to the area of the host to be treated where it will desirably release a therapeutically effective amount of the agent to the affected area. For the administration of systemic therapeutically active agents, delivery device 10 can be applied to any area of the patient's skin. Delivery device 10 can also be applied to a mucosal surface, such as the oral mucosa. Delivery device 10 may be replaced as desired, as is necessary to maintain a therapeutically effective blood level of the therapeutically active agent or as is convenient to the user. Delivery device 10 exhibits substantially sustained release of the therapeutically active agent such that a therapeutically effective blood level of the drug can be achieved and/or maintained for an extended period of time. Delivery device 10 can also be used to maintain a desired level of the therapeutically active agent in the vicinity of the area to which the delivery device is applied if the treatment being effected is desirably local rather than systemic.

The present invention will now be described in connection with the following illustrative examples.

### EXAMPLE 1

### Formation of Texturized Backing

An extrusion cast, thermoplastic, 3 mil (76 μm), polyethylene/vinyl acetate film (commercially available under the trade designation “CoTran 9720” from 3M) was mounted in roll form onto an unwind supply roller. The film was mounted under low tension effective to reduce undesirable wrinkling and stretching of the film. The film was threaded through a nip roll set-up containing a lower, heated, steel roller and an upper rubber roller. The rubber roller was biased against the steel roller at a pressure of 2.3 mPa. A continuous, stainless steel embossing belt was mounted on the heated, steel roller and a second “turn around” roller located below the heated roller. The second, turn around roller served to keep the embossing belt under tension and reduce buckling or other undesired movement of the embossing belt during the embossing operation. The embossing belt contained a microreplicated surface pattern of trihedral prisms as shown in FIGS. 2A and 2B. As the film passed through the nip roll set-up, the pattern on the embossing belt was correspondingly embossed onto the film.

During the embossing operation, the heated roller was maintained at a temperature of about 255° F. (124° C.). This caused the film to be heated to a temperature of about 210° F. (99° C.) to about 217° F. (103° C.). The film and belt each moved at a linear speed of 1.9 ft/min (0.58 m/min).

At the output end of the rubber roller/heated roller nip, an “air blow” knife was positioned to aid in cooling and removal of the patterned film from the embossing belt. The airflow directed toward such interface was at ambient temperature and was adjusted as needed to achieve good separation between the embossed film and the belt.

Upon removal from the embossing belt, the embossed film passed over a spreading roller to prevent formation of wrinkles as the film was wound upon a take-up roller.

### EXAMPLE 2

### Flame-Embossed Coarse Texturized Backing

Extrusion cast thermoplastic polyethylene/vinyl acetate film (CoTran™ 9720, 3M, St. Paul, Minn.), 0.075 mm thick, was mounted in roll form onto an unwind supply roller under low tension to reduce wrinkling and stretching during subsequent processing. The film was unwound at 110 m/min and exposed to the heat of a combustion burner while in contact with a hard elastomer backing roll heated at 38° C. In this process, the film was heated to a deformable but not molten state, and was passed into a nip at 610 kPa (88 psi) pressure between a male-patterned metal embossing roll cooled to 18° C. and a hard elastomer backing roll so that a pattern was embossed into the film. The embossed film was passed over chill rolls (18° C.) to cool the film to 23° C. prior to winding on a takeup roll.

The embossing roll produced a pattern of three-dimensional pyramid-shaped features as shown in FIG. 4, having raised areas on one side of the film (positive pattern) and indentations on the opposite side (negative pattern). The pattern included 36 lines per 2.54 cm in both the machine and crossweb directions.

### EXAMPLE 3

### Flame-Embossed Fine Texturized Backing

In the manner described in Example 2, a texturized film having a pyramidal pattern comprising 175 lines per 2.54 cm in each of the machine and crossweb directions was prepared from the CoTran™ 9720 film. This film also had raised areas on one side and complementary indentations on the other side of the film.

### EXAMPLE 4

### Film Swelling Evaluation

Test Method

FIG. 7 shows test fixture 200 used to evaluate film swelling. A film sample 202 measuring 2.5 cm×12.7 cm was cut from both the machine direction (MD) and cross-web direction (CD) of film 202 (shown in swelled state). Each sample was secured in a 2.5 cm×7.6 cm fixture 200 with the flat or (negative side, as the case may be) down, under sufficient lateral tension to flatten the film against bottom plate 204 of fixture 200. Fixture 200 was closed and film 202 was clamped in place, using holders 206 and 208. Excess film was trimmed from each end. A baseline thickness measurement was obtained, using attached micrometer 210. The entire fixture 200 was then lowered into shallow trough 212 filled to approximately 1.3 cm depth with swelling agent 214 and allowed to equilibrate for 30 minutes. While film 202 was still immersed, a second thickness measurement was obtained, with care taken to measure just to the top of swelled film 202 without deformation. The difference in thickness was calculated, and the value squared and converted to a percentage swelling. Each determination was repeated twice.

Test Results

Each of the three films described in Examples 1 through 3 was examined and compared with a commercial backing film sample, CoTran™ 9720 (3M, St. Paul, Minn.), that was not texturized. The swelling agent was isopropyl myristate. The results are shown in Table 1.

TABLE 1

| | Web | Percent |
|---|---|---|
| Sample | Direction | Swelling |
| CoTran ™ | MD | 3.2 |
| 9720 | | |
| Comparative | | |
| CoTran ™ | CD | 1.7 |
| 9720 | | |
| Comparative | | |
| Example 1 | MD | 1.7 |
| Example 1 | CD | 0.9 |
| Example 2 | MD | 3.5 |
| Example 2 | CD | 1.1 |
| Example 3 | MD | 2.9 |
| Example 3 | CD | 1.2 |

The results shown in Table 1 verify that isopropyl myristate is a swelling agent for these film samples. The next example shows that swelling may be camouflaged by samples of the present invention.

### EXAMPLE 5

### Film Swelling by Placebo Formulation

A placebo formulation containing 25% isopropyl myristate was compounded using a standard pharmaceutical grade acrylate pressure sensitive adhesive comprising a 75:5:20 (by weight) copolymer of isooctyl acrylate:acrylamide:vinyl acetate, prepared as described in U.S. Pat. No. 5,614,210, column 8 line 35, incorporated herein by reference. A 25% solids solution of the adhesive in ethyl acetate was prepared, then mixed with an excipient, USP grade isopropyl myristate (IPM), to equal 25% of the amount of adhesive solids. The formulation was roller mixed for 4 hours to incorporate the excipient completely. A 0.6 mm (24 mil) thick wet coating of the mixture was coated on 15 cm wide silicone-coated clear polyester release liner (Akrosil™ H3G, Akrosil, Menasha, Wis.) then dried at 49° C. for 30 minutes to remove excess solvent while retaining as much IPM as possible. The coated adhesive on the release liner was then cut into 6-15 cm×15 cm pieces. Embossed film samples described in Examples 1-3 were hand laminated to an adhesive piece with machine direction of each sample in the same direction, such that either the positive or negative sides of each backing material was in direct contact with the adhesive/excipient mixture. The samples were rolled down with a small hand roller to ensure contact with the IPM-filled adhesive and then allowed to stand at room temperature for specified intervals prior to evaluation. Results of the evaluations are shown in Table 2. In Table 2, “positive” and “negative” refer to the side of the film that faced away from the adhesive/excipient mixture. The comparative CoTran™ 9720 film exhibited distinct matte finishes on each side; “positive” refers to a coarser matte finish and “negative” refers to a finer matte finish. “Tunneling” describes elongated lifted areas resulting from smaller bubbles coalescing over time.

| Sample | Start | 4 Hr Exposure | 24 Hr Exposure |
|---|---|---|---|
| CoTran ™ 9720, | Flat | Tunnels and | Tunnels and |
| Positive | | Lifting | Lifting |
| Comparative | | | |
| CoTran ™ 9720, | Flat | Tunnels and | Tunnels and |
| Negative | | Lifting | Lifting |
| Comparative | | | |
| Example 1 Positive | Flat | Small bubbles | Slight wrinkling, |
| | | at interface | lifting from some |
| | | | bubbles |
| Example 1 Negative | Flat | Small bubbles | Small wrinkles, |
| | | at interface | slight lifting from |
| | | | bubbles, minor |
| | | | tunneling |
| Example 2 Positive | Small bubbles | Small bubbles | Slight lifting from |
| | at interface | at interface | some bubbles |
| Example 2 Negative | Flat | Flat | Flat |
| Example 3 Positive | Small bubbles | Small bubbles | Slight lifting from |
| | at interface | at interface | bubbles, minor |
| | | | tunneling |
| Example 3 Negative | Flat | Small bubbles | Slight lifting from |
| | | at interface | bubbles |

The data of Table 2 show that a coarse pattern, in direct contact with a potentially swelling excipient, successfully resisted or masked wrinkling due to swelling of the film. In practice, the size of the pattern can be optimized to minimize wrinkling, as a function of the degree of swelling exhibited by the film/excipient combination.

What is claimed is:

1. A therapeutic agent delivery device that can be applied onto a host in order to therapeutically administer a therapeutically active agent, or prodrug form thereof, to the host, said device comprising:
(a) a repository comprising a releasably stored dosage of the therapeutically active agent; and
(b) a backing protecting the repository, wherein the backing comprises inner and outer, opposed major surfaces, and further wherein at least one of said surfaces is texturized and comprises a plurality of three-dimensional topographic features.

2. The device of claim 1, wherein the inner major surface is texturized.

3. The device of claim 1, wherein the outer major surface is texturized.

4. The device of claim 1, wherein both major surfaces are texturized.

5. The device of claim 1, wherein the texturized surface comprises a microreplicated pattern.

6. The device of claim 5, wherein said pattern comprises an array of trihedral prisms.

7. The device of claim 1, wherein at least substantially all of the texturized surface bears a texture comprising features having a height in the range from 50 micrometers to about 1000 micrometers and a breadth in the range from about 0.1 micrometers to about 3 millimeters.

8. The device of claim 1, wherein the texturized surface comprises features of sufficiently small size such that individual features are not visually discernible, but the texturized surface has a visually discernible matte finish.

9. The device of claim 1, wherein the texturized surface comprises features of a sufficiently large size such that individual features are visually discernible.

10. The device of claim 1, wherein the device comprises a penetration enhancer.

11. The device of claim 10, wherein the penetration enhancer comprises a fatty acid or fatty acid ester.

12. The device of claim 10, wherein at least a portion of the penetration enhancer is incorporated into the backing, and wherein the three dimensional topographic features help to reduce swelling of the backing as compared to an otherwise identical device having a backing that is substantially free of said three dimensional topographic features.

13. A therapeutic agent delivery device that can be applied onto a host in order to therapeutically administer a therapeutically active agent, or prodrug form thereof, to the host, said device comprising:
(a) a repository comprising a releasably stored dosage of the therapeutically active agent; and
(b) a backing protecting the repository, wherein the backing comprises inner and outer, opposed major surfaces, at least one of said surfaces comprising a plurality of three-dimensional topographic features in a manner effective to increase the vapor transmission rate of the backing as compared to an otherwise identical backing lacking such features.

14. A therapeutic agent delivery device that can be applied onto a host in order to therapeutically administer a therapeutically active agent, or prodrug form thereof, to the host, said device comprising:
(a) a repository comprising a releasably stored dosage of the therapeutically active agent; and
(b) a backing protecting the repository, wherein the backing comprises inner and outer, opposed major surfaces, at least one of said surfaces comprising a plurality of three-dimensional topographic features of a size and density effective to improve the ability of the backing to camouflage wrinkles in the backing as compared to an otherwise identical backing lacking such features.

15. A therapeutic agent delivery device that can be applied onto a host in order to therapeutically administer a therapeutically active agent, or prodrug form thereof, to the host, said device comprising:
(a) a repository comprising a releasably stored dosage of the therapeutically active agent; and
(b) a backing protecting the repository, wherein the backing comprises inner and outer, opposed major surfaces, at least one of said surfaces comprising a plurality of three-dimensional topographic features effective to impart a visually discernible texture to at least a majority of the surface.

16. A method of therapeutically treating a host comprising applying a therapeutic agent delivery device to a desired treatment site on the host wherein the therapeutic agent delivery device comprises:
(a) a repository comprising a releasably stored dosage of the therapeutically active agent, or prodrug form thereof; and
(b) a backing protecting the repository, wherein the backing comprises inner and outer, opposed major surfaces, wherein at least one of said surfaces is texturized and comprises a plurality of three-dimensional topographic features.

17. A method of making a drug delivery device, comprising the steps of:
(a) forming a therapeutic agent repository, said repository storing a dosage of a therapeutically active agent, or prodrug form thereof; and
(b) causing the therapeutic agent repository to be positioned in a therapeutic agent delivery device, said device comprising a texturized backing layer.
